(19) United States
(12) Patent Application Publication
(10) Pub. No.: US 2003/0231993 A1
(43) Pub. Date: Dec. 18, 2003

(54) Car air cleaning apparatus

(76) Inventor: Jason Yan, Taipei (TW)

Correspondence Address: Jason Yan, P.O. Box 26-757, Taipei, 106, TW

(21) Appl. No.: 10/172,449

(22) Filed: Jun. 12, 2002

(51) Int. Cl. A61L 9/00; A62B 7/08; A61L 2/00

(52) U.S. Cl. 422/305; 422/306; 422/123; 422/124; 422/24

(57) ABSTRACT

A car air cleaning apparatus of the invention includes a power charging plug mounted to a casing. A front side and a rear side of the casing are respectively provided with an air outlet and an air inlet. The casing is further internally provided with an ultraviolet lamp and a fan. Via a rotation of the fan, an external air flows in the casing through the air inlet, is subjected to deodorization and disinfecting actions by means of the ultraviolet lamp, and flows out through the air outlet to provide the interior of the car with a clean air. The casing is further internally provided with a set of power charging batteries to provide power, the batteries being electrically connected to the power charging plug.

### BACKGROUND OF THE INVENTION

1. Field of the Invention

The invention relates to a car air cleaning apparatus and, more particularly, to a car air cleaning apparatus that is internally provided with a power supply, and is further conveniently transportable and easily mounted within a car.

2. Description of the Prior Art

As the number of cars and motorcycles rapidly increases, air pollution due to gas emission affects human health by causing various chronic diseases such as asthma. Therefore, car drivers may additionally install an internal air filter within their car. The air filter may be also optionally provided in the car when it is originally purchased. The air filter allows to maintain a relatively clean air within the car so that the driver is not affected by the external polluted air.

However, the air filter as conventionally provided is usually directly electrically connected to the power source of the car. Therefore, if the power source of the car is turned off, no operating power is supplied to the air filter that, consequently, cannot operate. Furthermore, not every car is provided with an air filter. In these situations, the driver thus has to endure an exposure to polluted air, which negatively affect his/her health.The traditional air filter therefore needs to be improved.

### SUMMARY OF THE INVENTION

It is therefore an object of the invention to provide a car air cleaning apparatus that is internally provided with a power supply so as to maintain a normal air cleaning operation even if the power source of the car is turned off.

It is another object of the invention to provide a car air cleaning apparatus that is conveniently transportable, easily installed, and suitable for being mounted in cars that are not provided with air filters.

To accomplish the above and other objectives, a car air cleaning apparatus of the invention comprises a power charging plug mounted to a casing. A front side and a rear side of the casing are respectively provided with an air outlet and an air inlet. The casing is further internally provided with an ultraviolet lamp and a fan. Via a rotation of the fan, an external air flows in the casing through the air inlet, is subjected to deodorization and disinfecting actions by means of the ultraviolet lamp, and flows out through the air outlet to provide the interior of the car with a clean air. The casing is further internally provided with a set of power charging batteries to provide power, the batteries being electrically connected to the power charging plug.

To provide a further understanding of the invention, the following detailed description illustrates embodiments and examples of the invention, this detailed description being provided only for illustration of the invention.

### BRIEF DESCRIPTION OF THE DRAWINGS

The drawings included herein provide a further understanding of the invention. A brief introduction of the drawings is as follows:

FIG. 1 is an exploded view of a car air cleaning apparatus according to an embodiment of the invention;

FIG. 2 is an external perspective view of a car air cleaning apparatus according to an embodiment of the invention; and

FIG. 3 is a sectional view of a car air cleaning apparatus according to an embodiment of the invention.

### DETAILED DESCRIPTION OF THE EMBODIMENT

Wherever possible in the following description, like reference numerals will refer to like elements and parts unless otherwise illustrated.

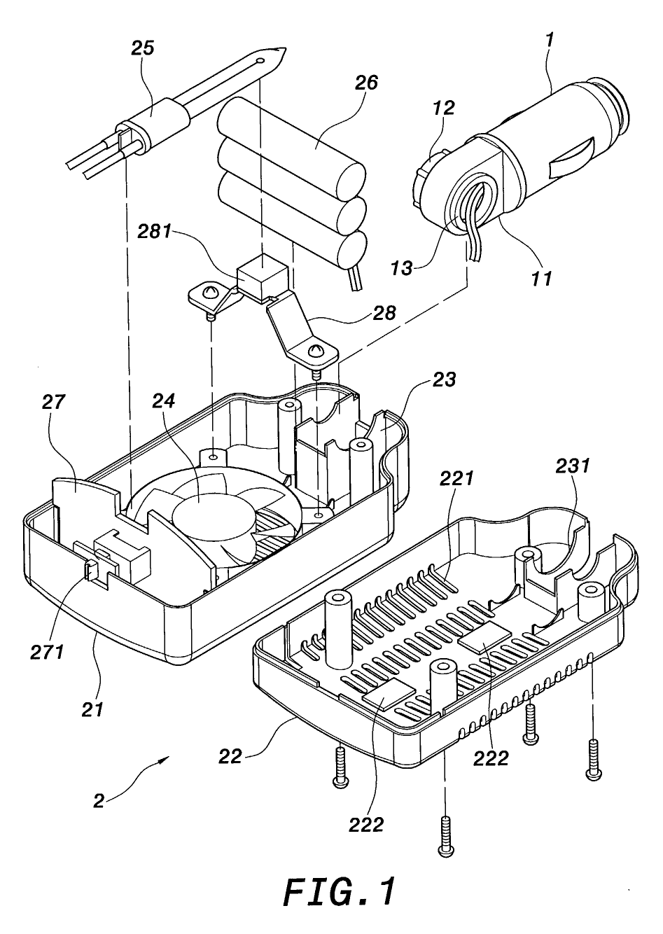

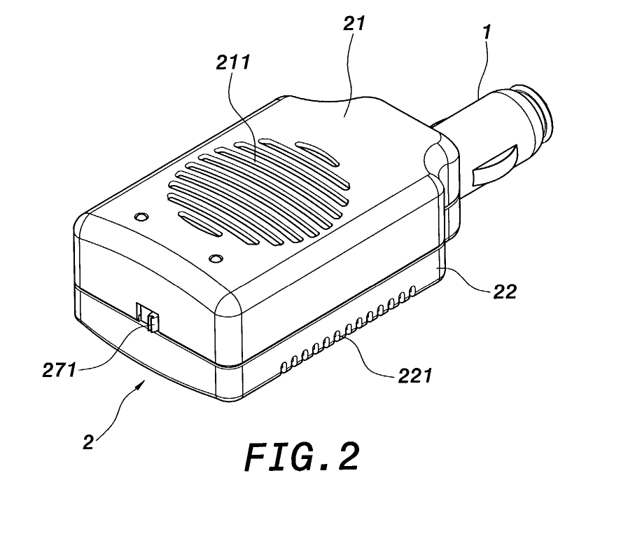

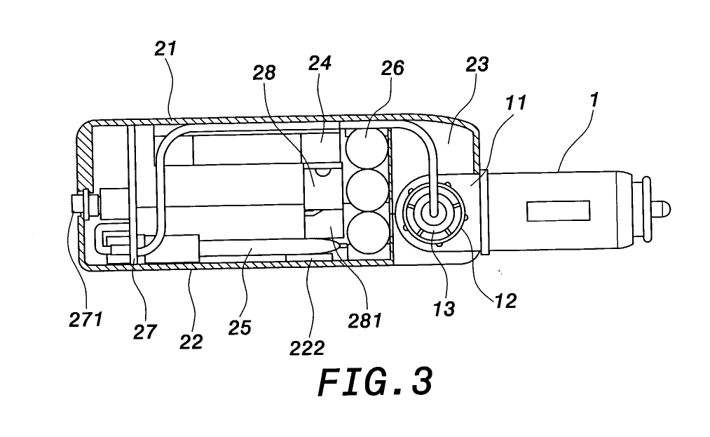

Referring to FIG. 1 through FIG. 3, the invention provides a car air cleaning apparatus that comprises a power charging plug 1 and a casing 2.

The power charging plug 1 is used to electrically connect a power source in the car, such as a cigar lighter socket. A side of the power charging plug 1 is provided with an extension arm 11 at two lateral sides of which are respectively disposed a roller 12 and a protruding ring 13.

The casing 2 is comprised of a front cover 21 and a rear cover 22 that are assembled with each other via screw elements. The front and rear covers 21, 22 respectively have inner sidewalls at an end portion that define a receiving base 23. The casing 2 is mounted with the power charging plug 1 with the extension arm 11 rotatably arranged on the receiving base 23 and the roller 12 and protruding ring 13 respectively positioned on two arcuate edges 231 at two respective sides of the receiving base 23. The casing 2 is thereby enabled to effectuate an upward/downward rotary adjustment with respect to the extension arm 11. A front side and a rear side of the casing 2 are further respectively provided with an air outlet 211 and an air inlet 221. On the front and rear covers 21, 22, the air outlet 211 is comprised of a plurality of grooves that are approximately arranged in a disk shape, while the air inlet 221 is comprised of a plurality of grooves that are approximately arranged in a rectangle shape.

The casing 2 is internally provided with a fan 24, an ultraviolet lamp 25, a set of power charging batteries 26, and a control circuit board 27. The fan 24 is fastened on an inner surface of the front cover 21 proximate to the air outlet 211 by means of an attachment frame 28. The ultraviolet lamp 25 is attached on an inner surface of the rear cover 22 proximate to the air inlet 221 by means of a plurality of adhesive tapes 222. The attachment frame 28 is further attached to a protection pad 281 that abuts against the ultraviolet lamp 25, thereby reinforcing the positioning and fastening of the ultraviolet lamp 25. The power charging batteries 26 are disposed at an adequate position in the casing 2 and is electrically connected to the power charging plug 1 via the control circuit board 27. The power charging batteries 26 supply the fan 24 and the ultraviolet lamp 25 with necessary operating voltage. The control circuit board 27 is arranged at another end portion within the casing 2, and is sideways provided with a switch 271 that is externally exposed out of the casing 2. The control circuit board 27 charges the power charging batteries 26 with power, and is further capable of determining the amount of power stored in the power charging batteries 26 to provide an adequate power charging until the power charging batteries 26 are completely full, after which the power charging is automatically stopped.

In utilization, the power charging plug 1 is inserted in the power source socket of the car. By turning on the switch 271 of the casing 2, the fan 24 then rotates and the ultraviolet lamp 25 is lighted up. External air hence flows in the casing 2 through the air inlet 221, and via the ultraviolet irradiation from the ultraviolet lamp 25, the external air is subject to deodorization and disinfecting actions. Thereafter, the cleaned air flows out through the air outlet 211, achieving an air cleaning cycle. The interior cabin of the car hence is provided with a clean air. If the voltage supplied by the power charging batteries 26 is not sufficient, power charging can be achieved via the power source of the car to which is electrically connected the power charging plug 1. After the power charging batteries 26 are full, power charging is automatically stopped. The power charging batteries 26 can be therefore maintained in a state of sufficient power at any time. Even if the car power source is turned off, the air cleaning operation can still be maintained by means of the power charging batteries 26, provided the switch 271 is turned on.

As described above, the air cleaner for car therefore includes at least the following advantages:

(1) Via power charging batteries internally incorporated, the air cleaning apparatus of the invention can maintain a normal air cleaning operation even if the car power source is not turned on. When the voltage provided by the power charging batteries is too low, the batteries can be recharged via connection to the power source socket of the car.

(2) The air cleaning apparatus of the invention is lightweight and easily transportable. Furthermore, the mounting is easily achieved via connecting the power charging plug to the power source socket of the car. The air cleaning apparatus of the invention can be therefore installed within cars that are not provided with air filters.

It should be apparent to those skilled in the art that the above description is only illustrative of specific embodiments and examples of the invention. The invention should therefore cover various modifications and variations made to the herein-described structure and operations of the invention, provided they fall within the scope of the invention as defined in the following appended claims.

What is claimed is:

1. An air cleaning apparatus for car, electrically connected to a power source socket of the car, the air cleaning apparatus comprising:
a power charging plug;
a casing, mounted to the power charging plug, a front side and a rear side of the casing being respectively having an air outlet and an air inlet, the casing further having an ultraviolet lamp and a fan thereof, thereby an air flowing in the casing through the air inlet via a rotation of the fan, is subjected to deodorization and disinfecting actions by means of the ultraviolet lamp, and the air flowing out through the air outlet to provide the air being cleaned ; and
a set of power charging batteries electrically connected to the power charging plug being further provided within the casing to provide power.

2. The apparatus of claim 1, wherein the casing hasa front cover and a rear cover that are assembled with each other.

3. The apparatus of claim 2, wherein a portion of inner sidewalls of the front and rear covers forms a receiving base that rotatably receives an extension arm at one end of the power charging plug, the casing being thereby enabled to effectuate a rotary adjustment with respect to the power charging plug.

4. The apparatus of claim 2, wherein an attachment frame is fixedly connected with the fan which is fastened on an inner surface of the front cover and proximate to the air outlet .

5. The apparatus of claim 2, wherein at least one adhesive tape is attached onto the ultraviolet lamp which is proximate to a side of the air inlet .

6. The apparatus of claim 4, wherein a protection pad is attached on the attachment frame, the protection pad abutting against the ultraviolet lamp.
